(19) United States
(12) Patent Application Publication
(10) Pub. No.: US 2026/0240808 A1
(43) Pub. Date: Aug. 20, 2026

(54) A COMPOSITION OF A READY TO USE ASCORBIC ACID INJECTION AND A METHOD THEREOF

(71) Applicant: MAIVA PHARMA PRIVATE LIMITED, Hosur (IN)

(72) Inventors: Alagumurugan Alagarswamy, Hosur (IN); Bhaskar Krishna Arumugam, Bangalore North (IN)

(21) Appl. No.: 18/713,298

(22) PCT Filed: May 16, 2023
(86) PCT No.: PCT/IB2023/055018

§ 371 Date: May 24, 2024

(30) Foreign Application Priority Data
Jun. 7, 2022 (IN) 202241032599

(51) Int. Cl. A61K 31/375; A61K 9/00; A61K 9/08; A61K 47/02; A61K 47/18; A61L 2/022; A61L 103/10; B65B 3/00

(52) U.S. Cl. CPC A61K 31/375; A61K 9/0019; A61K 9/08; A61K 47/02; A61K 47/183; A61L 2/022; B65B 3/003; A61L 2103/10

(57) ABSTRACT

A composition 10 of a ready to use ascorbic acid injection 50 is provided. The composition includes 2.5 percentage by weight ascorbic acid 20. The composition also includes 0.00125 percentage by weight edetate disodium 30. The composition further includes 1.193 percentage by weight sodium bicarbonate 40.

### EARLIEST PRIORITY DATE

This application claims priority from a patent application filed in India having Patent Application No. 202241032599, filed on Jun. 7, 2022, and titled “A COMPOSITION OF A READY TO USE ASCORBIC ACID INJECTION AND A METHOD THEREOF.” and a PCT Application no. PCT/IB2023/055018 filed on May 16, 2023, and titled “A COMPOSITION OF A READY TO USE ASCORBIC ACID INJECTION AND A METHOD THEREOF.”

### FIELD OF INVENTION

Embodiments of the present disclosure relate to the field of medicinal preparations, and more particularly to a composition of a ready to use ascorbic acid injection and a method thereof.

### BACKGROUND

Ascorbic acid is necessary for growth, development, and repair of human tissues. Parenteral supplementation of the ascorbic acid may be necessary for treating patients suffering from scurvy, gastric disorders, and injuries. Supplementation of the ascorbic acid orally or through diet may not be possible in every instances. The ascorbic acid is indicated for short term (up to 1 week) treatment of scurvy in adult and paediatric patients, age 5 months and older, for whom oral administration is not possible, insufficient, or contraindicated. Recommended dose of ascorbic acid injection ranges from 50 milli gram (mg) to 200 mg once daily based on age of patient population.

At present, the ascorbic acid injection is supplied as a pharmacy bulk package vials having 25,000 mg per 50 milli litre (mL) ascorbic acid content. The pharmacy bulk package is intended to dispense single doses to multiple patients in a pharmacy admixture program and is restricted to preparation of admixtures for infusion. Concentration of the ascorbic acid in each vial is 500 milligrams per millilitre, making the drug in the vial hypertonic, approx. 5,900 milli osmol per litre.

Further, injection of the hypertonic drug may cause pain during administration. In order to avoid such a scenario, dilution of hypertonic drug solution with a suitable infusion solution is carried out before the administration. Also, once the pharmacy bulk package vial is pricked, entire drug in the vial needs to be used within four hours and any unused portion is otherwise discarded.

Moreover, the ascorbic acid injection is prepared with the aid of sodium bicarbonate or sodium carbonate to increase solubility of the ascorbic acid by forming sodium ascorbate. Mixture of the ascorbic acid and the sodium bicarbonate or sodium carbonate may create pressure inside the vial due to the formation of carbon dioxide gas over the shelf life period, especially when stored at room temperature. To retard the accumulation of pressure inside the vial and to maintain stability of the drug, the concentrate pharmacy bulk package vials need to be kept in refrigerated condition, further making the storage process complex.

Hence, there is a need for an improved composition of a ready to use ascorbic acid injection and a method thereof to address the aforementioned issue(s).

### BRIEF DESCRIPTION

In accordance with an embodiment of the present disclosure, a composition of a ready to use ascorbic acid injection is provided. The composition includes 2.5 percentage by weight ascorbic acid. The composition also includes 0.00125 percentage by weight edetate disodium. The composition further includes 1.193 percentage by weight sodium bicarbonate.

In accordance with another embodiment of the present disclosure, a method for preparing a ready to use ascorbic acid injection is provided. The method includes mixing 0.00125 percentage by weight edetate disodium, 1.193 percentage by weight sodium bicarbonate and 90 percentage by weight water to obtain a first solution. The method also includes mixing 2.5 percentage by weight ascorbic acid and the first solution to obtain a second solution. The method further includes filtering the second solution by one or more filters to obtain the ready to use ascorbic acid injection.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a composition of a ready to use ascorbic acid injection in accordance with an embodiment of the present disclosure;

FIG. 2 is graphical representation of one embodiment of the FIG. 1, depicting variation in potency of the ready to use ascorbic acid injection with respect to time in accordance with an embodiment of the present disclosure; and

FIG. 3 is a flow chart representing the steps involved in a method of preparation of a ready to use ascorbic acid injection in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a composition of a ready to use ascorbic acid injection and a method thereof. The composition includes 2.5 percentage by weight ascorbic acid. The composition also includes 0.00125 percentage by weight edetate disodium. The composition further includes 1.1925 percentage by weight sodium bicarbonate.

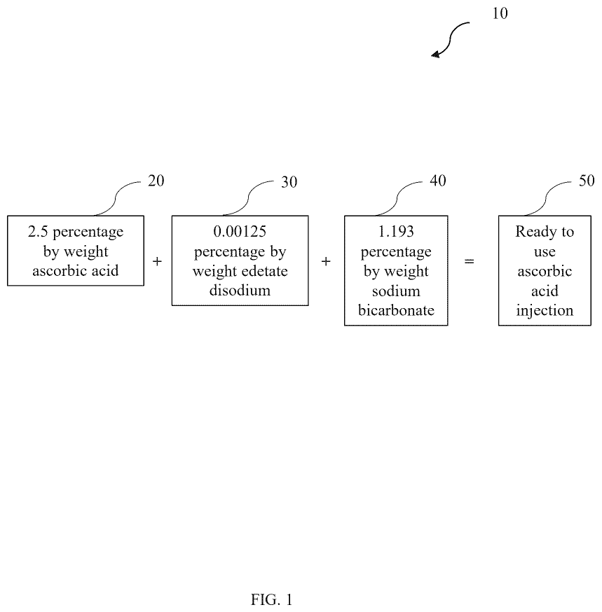

FIG. 1 is a block diagram representation of a composition 10 of a ready to use ascorbic acid injection 50 in accordance with an embodiment of the present disclosure. The composition 10 includes 2.5 percentage by weight ascorbic acid 20. In one embodiment, the ascorbic acid 20 may include concentration of 25 milligram per milli liter. In such an embodiment, osmolarity of the ascorbic acid 20 may be approximately 290 milliosmole per liter. The composition 10 also includes 0.00125 percentage by weight edetate disodium 30. In one embodiment, concentration of the edetate disodium 30 may be 0.0125 mg/mL. In one embodiment, the edetate disodium 30 may be adapted to shield the ascorbic acid from oxidative degradation by forming stable compounds with the metals. In such an embodiment, the edetate disodium 30 may be acting as a chelating agent.

Moreover, the composition 10 further includes 1.193 percentage by weight sodium bicarbonate 40. In one embodiment, concentration of the sodium bicarbonate 40 may be 11.93 mg/mL. In a specific embodiment, molar ratio between the ascorbic acid 20 and the sodium bicarbonate may be 1:1. In one embodiment, the sodium bicarbonate 40 may be replaced with 0.752 percentage by weight sodium carbonate. In such an embodiment, concentration of the sodium carbonate may be 7.52 mg/mL. In such an embodiment, mole ratio of the ascorbic acid 20 and the sodium carbonate may be 1:0.5 In one embodiment, the sodium bicarbonate 40 or sodium carbonate may be adapted to facilitate the dissolution and modify potential of hydrogen (pH) value of the composition 10. In some embodiments, the ascorbic acid 20 and the sodium bicarbonate 40 may include molar ratio of 1:1. In one embodiment, the composition 10 may include a potential of hydrogen value (pH) in a range between 5 to 7, In an exemplary embodiment, the composition may include a potential of hydrogen value (pH) in a range between 5.5 and 6.5.

Examples of different fill volumes of the composition 10 of the ready to use, ascorbic acid injection 50 covering the recommended dosage range of 50 mg to 200 mg is shown in Table 1.

TABLE 1
Various formulations of the composition of the ready to use ascorbic acid injection.

| | | | 50 mg/2 mL | 100 mg/4 mL | 200 mg/8 mL | |
|---|---|---|---|---|---|---|
| Sl. | | | Quantity in milli gram/vial | | | Percentage |
| No. | Ingredient | Function | (mg/vial) | | | w/v |
| 1 | Ascorbic acid | Drug substance | 50.0 | 100.0 | 200.0 | 2.50 |
| 2 | Edetate disodium, | Chelating agent | 0.025 | 0.050 | 0.10 | 0.00125 |
| | dihydrate | | | | | |
| 3 | Sodium bicarbonate | pH adjusting | 23.85 | 47.70 | 95.40 | 1.1925 |
| 4 | Sodium hydroxide | agents | Quantum satis (q.s) to adjust the pH about 6.3 | | | |
| 5 | Water for Injection | Vehicle | q.s. to | q.s. to | q.s. to | q.s. |
| | | | 2 mL | 4 mL | 8 mL | to 100.0% |

| | | | |
|---|---|---|---|
| 6 | Nitrogen | Processing | Not applicable |

aid

Further, from the table 1 it has been observed that the formulations of the ready to use ascorbic acid injection 50 may include, but not limited to, 50 mg/2 mL, 100 mg/4 mL and 200 mg/8 mL Ascorbic acid equivalent to 56.2 mg/2 ml, 112.4 mg/4 mL and 224.8 mg/8 mL of sodium ascorbate respectively. An inert gas, such as Nitrogen may be used to reduce the dissolved oxygen in the bulk solution and to displace headspace oxygen present in the vial. The sodium hydroxide may be used for adjustment of the pH of the ready to use ascorbic acid injection.

Variations in potential of hydrogen (pH) value of the ready to use ascorbic acid injection 50 with respect to variation in quantity of the sodium bicarbonate 40 is provided in Table 2.

TABLE 2
Variations in potential of hydrogen (pH) value of the ready to use ascorbic acid
injection with respect to variation in quantity of the sodium bicarbonate

| | | Formula | Formula | Formula | Formula | Formula | Formula |
|---|---|---|---|---|---|---|---|
| | | 1 | 2 | 3 | 4 | 5 | 6 |
| | | Ascorbic acid to sodium bicarbonate mole ratio | | | | | |
| | | | 1:0.5 | 1:0.75 | 1:0.9 | 1:1 | 1:1.2 |
| | | No sodium | mole | mole | mole | mole | mole |
| Sr. | | bicarbonate | ratio | ratio | ratio | ratio | ratio |
| No. | Ingredient | Quantity in mg/mL | | | | | |
| 1 | Ascorbic acid | 25.0 | 25.0 | 25.0 | 25.0 | 25.0 | 25.0 |
| 2 | Edetate disodium, | 0.0125 | 0.0125 | 0.0125 | 0.0125 | 0.0125 | 0.0125 |
| | dihydrate | | | | | | |
| 3 | Sodium bicarbonate | Not used | 5.97 | 8.95 | 10.74 | 11.93 | 14.32 |
| 4 | Water for Injection | q.s. to | q.s. to | q.s. to | q.s. to | q.s. to | q.s. to |
| | | 1 mL | 1 mL | 1 mL | 1 mL | 1 mL | 1 mL |

| | | |
|---|---|---|
| 5 | Nitrogen | Not applicable |

| | | | | | | |
|---|---|---|---|---|---|---|
| pH of the solution | About 2.4 | About 4.3 | About 4.7 | About 5.3 | About 5.9 | About 6.4 |

Also, from the table 2 it has been observed that potential of hydrogen (pH) value of the ready to use ascorbic acid injection 50 increases proportionately with addition of the sodium bicarbonate 40. Potency trend data of the various formulations with respect to time is provided in table 3.

TABLE 3
Potency trend data with respect to time
of the formulations with varying pH.

| | | Potency | | | |
|---|---|---|---|---|---|
| | | after 2 | | | |
| | | weeks | | | Potency |
| | | 40° C./75% | Potency after | | after |
| | | Relative | 4 weeks | | 6 weeks |
| | Initial | Humidity | 40° C./ | 25° C./ | at 40° C./ |
| Formulations | potency | (RH) | 75% RH | 60% RH | 75% RH |
| Formula 1 | 97.1 | 95.9 | 91.4 | 97.4 | 83.6 |
| (pH About | | | | | |
| 2.4) | | | | | |
| Formula 2 | 96.7 | 97.5 | 93.6 | 99.3 | 88.9 |
| (pH About | | | | | |
| 4.3) | | | | | |
| Formula 3 | 100.0 | 100.2 | 97.7 | 100.9 | 94.2 |
| (pH About | | | | | |
| 4.7) | | | | | |
| Formula 4 | 98.4 | 99.0 | 98.3 | 97.4 | 95.9 |
| (pH About | | | | | |
| 5.3) | | | | | |
| Formula 5 | 95.7 | 95.2 | 95.2 | 94.7 | 94.8 |
| (pH About | | | | | |
| 5.9) | | | | | |
| Formula 6 | 97.6 | 96.3 | 97.8 | 97.1 | 96.7 |
| (pH About | | | | | |
| 6.4) | | | | | |

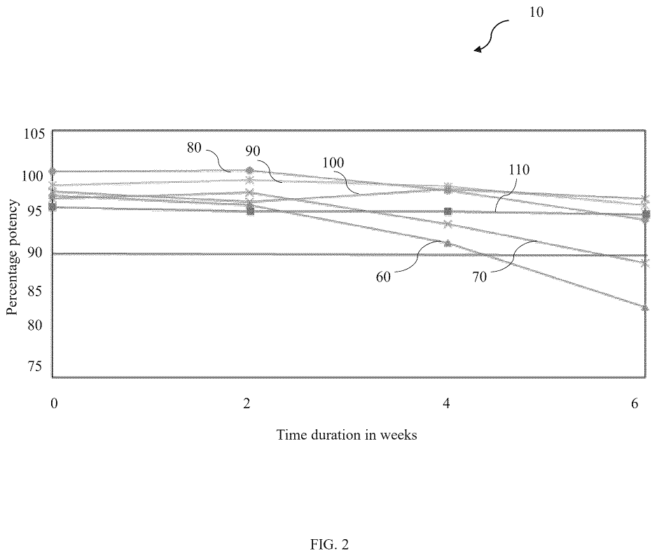

Additionally, from the table 3 it has been observed that stability of the ready to use ascorbic acid injection 50 is deteriorating significantly with time for the formulations having the potential of hydrogen values (pH) less than 4.7 and the formulations has satisfactory stability characteristics at pH about 6. Graphical representation of percentage variation of the potency of the ready to use ascorbic acid injection 50 having pH value 2.3 60, pH value 4.3 70, pH value 4.7 80, pH value 5.3 90, pH value 5.7 100, and pH value 6 110 with respect to time is shown in FIG. 2.

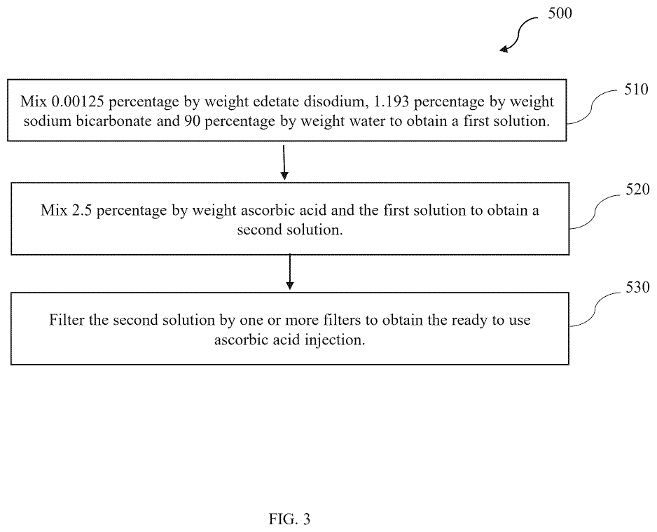

FIG. 3 is a flow chart representing the steps involved in a method 200 of preparation of a ready to use ascorbic acid injection in accordance with an embodiment of the present disclosure. The method 200 includes mixing 0.00125 percentage by weight edetate disodium, 1.193 percentage by weight sodium bicarbonate and 90 percentage by weight water to obtain a first solution in step 210. In one embodiment, concentration of the edetate disodium may be 0.0125 mg/mL. In some embodiments, concentration of the sodium bi carbonate may be 11.90 mg/ml. The method 200 also includes mixing 2.5 percentage by weight ascorbic acid and the first solution to obtain a second solution in step 220. In one embodiment, concentration of the ascorbic acid may be 25 mg/ml. In one embodiment, the ascorbic acid may include concentration of 25 milli gram per milli liter. In such an embodiment, osmolarity of the ascorbic acid may be about 290 milli osmole per liter. In one embodiment, the edetate disodium 30 may be adapted to shield ascorbic acid from oxidative degradation by forming stable compounds with metals. In such an embodiment, the edetate disodium 30 may be acting as a chelating agent. In one embodiment, the sodium bicarbonate and or sodium hydroxide may be adapted to modify potential of hydrogen (pH) value of the composition. In some embodiments, the ascorbic acid and the sodium bicarbonate may include molar ratio of about 1:1. In one embodiment, pH of the second solution may be adjusted by adding diluted sodium hydroxide or additional sodium bicarbonate followed by volume make upto batch volume.

The method 200 further includes filtering the second solution by one or more filters to obtain the ready to use ascorbic acid injection in step 230. In one embodiment, filtering the second solution by the one or more filters may include filtering the second solution by using sterilizing grade filters. In some embodiments, filling the ready to use ascorbic acid injection in amber type 1 glass vials. In such an embodiment, the type 1 glass vials may be either treated or untreated. In such an embodiment, the amber type 1 glass vials may be closed by a butyl rubber stopper. In a specific embodiment, adjusting potential of hydrogen (pH) value of the second solution may include adjusting the potential of hydrogen value using sodium bicarbonate or sodium hydroxide.

Various embodiments of the composition of a ready to use ascorbic acid injection and a method thereof described above enable various advantages. The ready to use ascorbic acid injection is isotonic and may be administered without further dilution. Due to the diluted nature of the ready to use ascorbic acid injection, the pressure build up inside the vials is found to be minimal which allows storage of the ready to use ascorbic acid injection at room temperature condition without any significant pressure build up, maintaining potency of the ready to use ascorbic acid injection. Capability of being stored in room temperate eliminates the need of refrigeration. The ready to use ascorbic acid injection is filled in vials matching standard single dosages such as 50 mg/2 ml, 100 mg/4 ml, 200 mg/8 mL thereby preventing wastage of the drug.

The single dose vial will give an opportunity to utilize the drug product more efficiently by avoiding wastage of the ready to use ascorbic acid injection and the same does not require any additional dilution or preparation steps which are required for the current concentrated reference listed drug (RLD) product supplied as pharmacy bulk package. Further, room temperature stable injection will give an opportunity to store and transport the ready to use ascorbic acid injection more efficiently.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. A composition of a ready to use ascorbic acid injection comprising:
2.5 percentage by weight ascorbic acid;
0.00125 percentage by weight edetate disodium; and
1.193 percentage by weight sodium bicarbonate

2. The composition as claimed in claim 1, wherein the ascorbic acid comprises concentration of 25 milli gram per milli liter.

3. The composition as claimed in claim 1, wherein the sodium bicarbonate and/or sodium hydroxide is adapted to modify potential of hydrogen (pH) value of the composition.

4. The composition as claimed in claim 1, wherein the edetate disodium is adapted to shield the ascorbic acid from oxidative degradation by forming stable compounds with metals.

5. The composition as claimed in claim 1, wherein the ascorbic acid and the sodium bicarbonate comprises molar ratio of about 1:1.

6. The composition as claimed in claim 1 comprises a potential of hydrogen value (pH) in a range between 5 to 7.

7. A method of preparation of a ready to use ascorbic acid injection comprising:
mixing 0.00125 percentage by weight edetate disodium, 1.1925 percentage by weight sodium bicarbonate and 90 percentage by weight water to obtain a first solution;
mixing 2.5 percentage by weight ascorbic acid and the first solution to obtain a second solution; and
filtering the second solution by one or more filters to obtain the ready to use ascorbic acid injection.

8. The method as claimed in claim 7, wherein filtering the second solution by the one or more filters comprises filtering the second solution by using sterilizing grade filters.

9. The method as claimed in claim 7, comprising filling the ready to use ascorbic acid injection in amber type 1 glass vials.

10. The method as claimed in claim 7, comprising adjusting potential of hydrogen (pH) value of the second solution comprises adjusting the potential of hydrogen value using sodium bicarbonate or sodium hydroxide.
